(19) United States
(12) Patent Application Publication
(10) Pub. No.: US 2010/0286524 A1
(43) Pub. Date: Nov. 11, 2010

(54) SYSTEM AND METHOD FOR ORGANIZING, RECORDING AND DISPLAYING IMAGES IN ULTRASOUND IMAGING SYSTEMS

(75) Inventors: Marco Daoura, Bothell, WA (US); Earl Canfield, Snohomish, WA (US); Wael Elseadiy, Sammammish, WA (US)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS, N.V., EINDHOVEN (NL)

(21) Appl. No.: 12/373,115

(22) PCT Filed: Jul. 6, 2007
(86) PCT No.: PCT/IB07/52665

§ 371 Date: Jan. 9, 2009

Related U.S. Application Data
(60) Provisional application No. 60/807,401, filed on Jul. 14, 2006.

(51) Int. Cl. A61B 8/14; A61B 5/04

(52) U.S. Cl. 600/443; 600/509

(57) ABSTRACT

An ultrasound imaging system includes a video processor receiving coherent echo signals from a beamformer. The video processor converts the coherent echo signals to image data, which are applied to a streaming digital video processor. The system also includes an ECG monitor and processor that obtains an ECG signal and uses the signal to provide heart cycle data indicative of the beginning and end of each of the plurality of heart cycles. The streaming digital video processor converts the image data into streaming digital data, which are divided into the heart cycle sections as indicated by the heart cycle data. The system also includes a digital video disk recorder that records a digital video disk containing the digital video data divided into chapters containing respective heart cycle sections. The recorder may also record chapters of ECG data derived from the ECG signal along with the digital video data.

This invention relates to diagnostic ultrasound imaging systems and, in particular, to a system and method for allowing ultrasound images to be organized and displayed in a manner that is related to the respective heart cycles during which the images were obtained.

One of the advantages that diagnostic ultrasound has had over many other diagnostic imaging modalities is the ability to produce realtime images. The advantage has been especially significant in cardiology where the physiology of a continually moving organ, the heart, are the subject of study. Realtime imaging has been a virtual necessity in echocardiography as compared with abdominal and obstetrical applications where the tissues and organs being studied are stationary and may be readily examined by static imaging. Echocardiologists, like other practitioners of diagnostic ultrasound, make records of their ultrasound examinations for subsequent diagnosis, review, and comparison. Since echocardiography studies use realtime ultrasonic imaging, they are conventionally recorded on videotape with a VCR, rather than being recorded statically on film or as photographic prints. A VCR has been an essential accessory for an echocardiography system for many years. More recently, it has been possible to stream digital data in real time as images corresponding to the data are obtained. This digital data can be saved by storing the data on an optical disk such as a digital video disk (“DVD”), which is capable of recording large volumes of data, such as image data generated during an ultrasound examination.

The advantage of being able to digitally record the large volume of image data generated during an ultrasound examination carries with it the disadvantage of making it difficult to organize the data for subsequent analysis of the images represented by the data. More specifically, the large volume of image data obtained during, for example, a cardiac ultrasound examination, makes it difficult to find images having specific characteristics or images that were taken during a specific cardiac event. Therefore, the large volume of data makes it even more important to be able to organize the data so that it can be subsequently accessed in a meaningful way. Also, although video cassette tapes can store a large volume of digital data, the organization of stored data for easy access is really not an issue because such tapes are can store data only in a linear manner, thus making random access to such data impossible regardless of how the data is organized. DVDs, on the other hand, allow random access to any portion of the stored data, but this benefit cannot be taken advantage of unless the data is organized in a meaningful way. Unfortunately, DVDs, being designed primarily for multimedia use, have very limited flexibility for organizing data, thus making it difficult to organize in a logical manner the ultrasound images stored on a DVD.

There is therefore a need for an ultrasound imaging system that can store image data on a DVD in an organized manner that readily allows images to be subsequently display in a logical manner.

A system and method of obtaining and recording an ultrasound image uses an ECG monitor to provide an ECG waveform that is processed to identify each of a plurality of heart cycles. Ultrasound is transmitted into a region of interest during at least some of the plurality of heart cycles, and resulting ultrasound echoes are received. These ultrasound echoes are converted into corresponding sets of echo signals, which are then delayed and summed to form coherent echo signals. A plurality of sets of the coherent echo signals, each corresponding to a portion of the same image, are processed to form respective sets of digital image data corresponding to a respective ultrasound image obtained during respective heart cycles. The digital image data are then divided into sections identified using the ECG waveform so that each section of digital image data corresponds to a respective ultrasound image obtained during a respective one of the plurality of heart cycles. The digital image data may be coupled to a digital video disk recorder, which records the digital image data on a digital video disk. The digital image data are recorded on the disk in chapters corresponding to the respective sections into which the digital image data was divided. Data corresponding to the ECG waveform divided into respective chapters may also be recorded on the digital video disk along with the corresponding chapters of the digital image data.

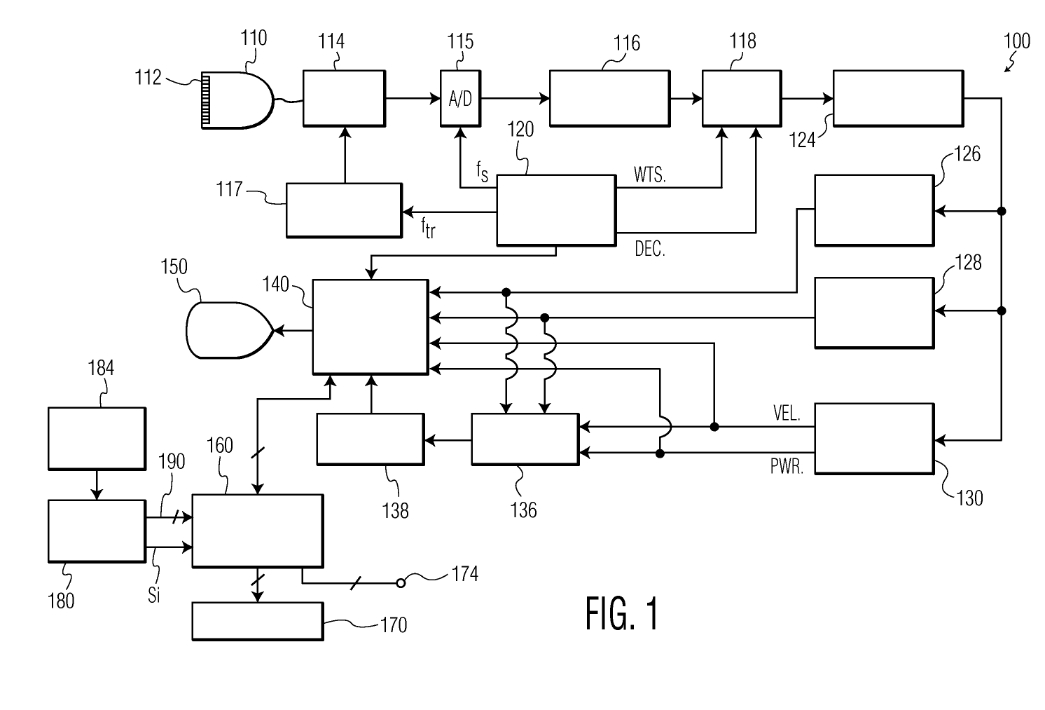

FIG. 1 is a block diagram of an ultrasound imaging system according to one example of the invention.

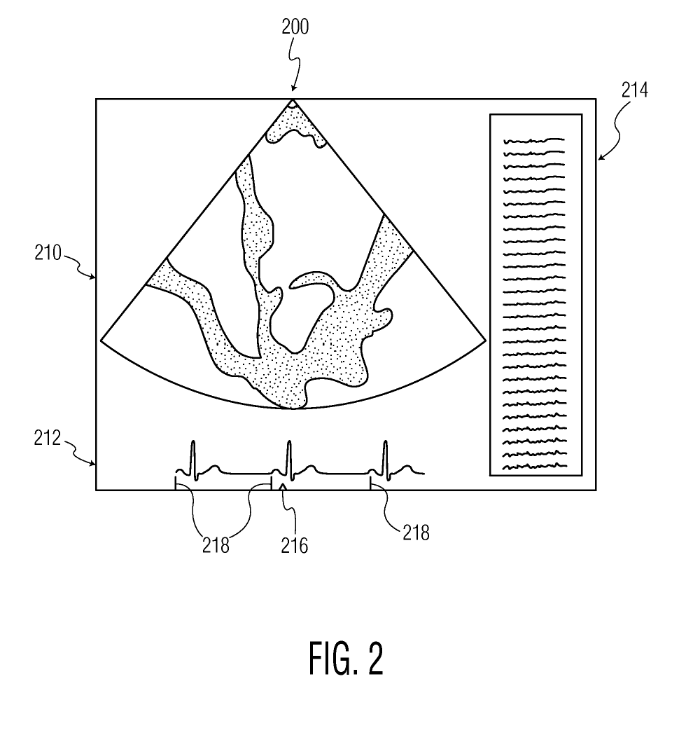

FIG. 2 is a screen shot showing one example of an ultrasound image, ECG waveform and chapter index displayed using the ultrasound imaging system of FIG. 1.

A diagnostic ultrasound imaging system 100 according to one example of the invention is shown in FIG. 1. The system 100 includes an ultrasound probe 110 having an array of transducer elements 112 which, in one example, is a one-dimensional line array of transducer elements 112 that can be used to examine a planar region beneath the probe 110. In another example of the invention, the transducer elements 112 are arranged in a two-dimensional array that can be used to examine a volumetric region beneath the probe 110.

The transducer elements 112 in the probe 110 are connected through a transmit/receive (“T/R”) switch 114 to a transmitter 117. The transmitter 117 is connected to a central controller 120, which causes the transmitter 117 to output an ultrasound signal at a desired time, transmit frequency fₜᵣ, and duration. The transmitter 117 applies the ultrasound signal through the T/R switch 114 to the transducer elements 112 in the probe 110. The transducer elements 112 then emit ultrasound into a planar or volumetric region beneath the probe 110. The transmitter 117 may also adjust the relative phases of the signals applied to the transducer elements 112 to either steer the transmitted ultrasound in a desired direction and/or to focus the transmitted ultrasound to a desired depth.

The transmitted ultrasound is reflected from anatomical features in the linear or volumetric region being examined, and the reflected ultrasound echoes are coupled to the transducer elements 112. The transducer elements 112 then produce corresponding echo signals, which are coupled through the T/R switch 114 and digitized by an analog-to-digital (“A/D”) converter 115. The sampling frequency fₛ of the A/D converter 115 is controlled by the central controller 120. The desired sampling rate dictated by sampling theory is at least twice the highest frequency of the received passband of the echo signals. Sampling rates higher than the minimum requirement are also desirable.

The echo signal samples from the individual transducer elements 112 are delayed and summed by a digital beamformer 116 to form coherent echo signals. The digital coherent echo signals are then filtered by a digital filter 118. In one example of the system 100, the transmit frequency fₜᵣ may be different from the passband frequency of the digital filter so that the digital filter 118 can pass signals having a frequency that is a harmonic of a fundamental frequency fₜᵣ of the transmitted ultrasound. The received echo signals may then be further processed, for instance by processing to remove artifacts such as speckle, by a digital signal processor 124.

The echo signals, after being filtered by the digital filter 118 and processed by the digital signal processor 124, are detected and processed by either a B mode processor 126 or a contrast signal detector 128 for display as a two or three dimensional ultrasonic image. The echo signals are also coupled to a Doppler processor 130 for Doppler processing to produce velocity and power Doppler signals which may be used to produce a colorflow, spectral, or power Doppler 2D image. The outputs of these processors 126, 128, 130 are also coupled to a 3D image rendering processor 136 for the rendering of three dimensional images, which are stored in a 3D image memory 138. Three dimensional rendering may be performed as described in U.S. Pat. No. 5,720,291, and in U.S. Pats. Nos. 5,474,073 and 5,485,842, the latter two patents illustrating three dimensional power Doppler ultrasonic imaging techniques. The signals from the B-mode processor 126, the contrast signal detector 128, and the Doppler processor 130 are coupled to a video processor 140 where they may be selected for two or three dimensional display on an image display 150 as dictated by user selection. The persistence of the displayed image and possibly other display parameters are controlled by the central controller 120.

The video processor 140 is also coupled to a streaming digital video processor 160 which processes image signals corresponding to signals received from the B-mode processor 126, the contrast signal detector 128 or the Doppler processor 130. More specifically, the streaming digital video processor 160 converts the image signals to streaming digital data in a desired digital video format. For example, the streaming digital data may be compressed to mpeg-2 format, which is suitable for recording on a DVD recorder 170. The streaming digital video processor 160 may output the streaming digital data to an external data port 174 as well as to the DVD recorder 170. Images corresponding to the streaming digital data can then be viewed by an external device (not shown) in real time or recorded by an external device (not shown) for subsequent viewing.

The streaming digital video processor 160 also receives an indexing signal Sᵢ from an ECG processor 180, which is, in turn, connected to an ECG monitor 184. The streaming digital video processor 160 uses the indexing signal Sᵢ from the ECG processor 180 to divide the streaming digital data into sections so that each section contains digital data for a respective heart cycle. The location in the heart cycles where each section begins and ends is controlled by the central controller 120. The ECG processor 180 also outputs on bus 190 ECG data indicative of an ECG waveform obtained by the ECG monitor 184. The streaming digital video processor 160 converts the ECG data into streaming ECG data, which is divided into the sections so that each section contains ECG data for the portion of the ECG waveform obtained during a respective heart cycle. The sections of streaming digital data and the sections of streaming ECG data are associated with each other so that the ultrasound image and ECG waveform obtained during the same heart cycle can be displayed together.

When the streaming digital data and streaming ECG data are applied to the DVD recorder 170, the DVD recorder 170 records a DVD with a large number of chapters, each of which contains digital data for a respective section of streaming digital data and a respective section of streaming ECG data. The DVD may also contain audio indicative, for example, of Doppler sounds or containing comments of a sonographer conducting the ultrasound examination from which the images were obtained. Further, as is well-known in the art, the images recorded on the DVD can be viewed with an index listing the chapters sequentially numbered. Using this index, a chapter containing an image recorded during a cardiac or other event can be easily located for subsequent viewing. Further, by recording the ultrasound images on a DVD, the images can be stored in a patient's chart in the form of the DVD. Also, the images may be viewed on any device that is capable of playing DVDs, such as personal computers and common DVD players. Although the streaming digital data is described as being recorded on a DVD, it will be understood that it also may be recorded on other and subsequently developed digital video recording media such as, for example, high definition digital video disks (“HDDVDs”) and the like.

An example of an ultrasound image recorded on a DVD by the DVD recorder 170 is shown on a screen 200 of the display 150 (FIG. 1) in FIG. 2. The screen 200 is divided into three sections: an image display section 210, an ECG waveform portion 212, and an index portion 214. The image display section 210 displays an ultrasound image, which can be any type of ultrasound image that the ultrasound system 100 is capable of generating. For example, the displayed image can be a two-dimensional B-mode image, a three-dimensional volumetric image, a Doppler image, to name a few.

With further reference to FIG. 2, the ECG waveform portion 212 displays in streaming fashion an ECG waveform for a number of heart cycles, which, in the example shown in FIG. 2, is for three heart cycles. Also displayed in the ECG waveform portion 212 is an image cursor 216. The image cursor 216 shows the location in one of the displayed heart cycles during which the displayed image was obtained. As mentioned above, the central controller 120 selects the location in the heart cycle where each chapter begins and ends. For this purpose, the central controller 120 controls the location of chapter cursors 218, which show the beginning and end points of the chapters.

Finally, the index portion 214 contains a listing of chapters on the DVD, each of which corresponds to a respective heart cycle. In the probable event that there are too many recorded chapters to be displayed in the index portion 214, the chapter corresponding to the heart cycle shown in the center of the ECG waveform portion 212 is located at the center of the chapter listing in the index portion 214. However, other chapter indexing formats may also be used.

The ultrasound imaging system 100 is therefore capable of recording a vast amount of image data in a manner that is organized to permit any heart cycle and desired image to be easily located. Further, when reviewing the ultrasound images, any image of significance can be easily noted for subsequent review by simply noting the corresponding chapter. Finally, the ultrasound images recorded by the system 100 are stored on a DVD where they can be viewed by readily available playback devices.

Although the present invention has been described with reference to the disclosed embodiments, persons skilled in the art will recognize that changes may be made in form and detail without departing from the spirit and scope of the invention. Such modifications are well within the skill of those ordinarily skilled in the art. Accordingly, the invention is not limited except as by the appended claims.

1.-11. (canceled)

12. A method of organizing and storing digital data corresponding to an ultrasound image of an anatomical structure of a patient, the method comprising:
obtaining an ECG waveform from the patient;
using the ECG waveform to identify each of a plurality of heart cycles of the patient;
dividing the digital data into sections each of which corresponds to a respective ultrasound image obtained during a respective one of the identified heart cycles; and
recording the digital data on a digital video disk, the digital data being recorded on the digital video disk in chapters corresponding to the respective sections into which the digital data are divided.

13. The method of claim 12, further comprising adjusting the beginning and ending points of each of the plurality of heart cycles with respect to the ECG waveform.

14. The method of claim 12 wherein the digital data to which the ultrasound image corresponds comprises a two-dimensional image.

15. The method of claim 12 wherein the digital data to which the ultrasound image corresponds comprises a three-dimensional volumetric image.

16. A method of displaying an ultrasound image, comprising:
providing a digital video disk storing digital data corresponding to a plurality of ultrasound images, and an ECG waveform indicative of a plurality of heart cycles occurring during a period of time that the digital data corresponding to the plurality of ultrasound images were obtained, the digital data being divided into a plurality of chapters each of which contains the stored digital data obtained during a respective one of the heart cycles;
reading the stored digital data from the digital video disk; and
using one of the chapters of digital data read from the digital video disk to display one of the ultrasound images and the ECG waveform corresponding to one of the read chapters of digital data.

17. The method of claim 16, further comprising displaying an index containing respective index entries for at least some of the chapters of digital data stored on the digital video disk, the index including an index entry for at least the chapter of digital data corresponding to the displayed ultrasound image and ECG waveform.

18. The method of claim 17, further comprising:
selecting an index entry for one of the chapters in the displayed index;
reading the digital data in the chapter indicated by the selected index entry; and
using the read digital data in the chapter indicated by the selected index entry to display the corresponding ultrasound image and ECG waveform.

19. The method of claim 16, further comprising displaying an image cursor adjacent the ECG waveform indicative of the time in a heart cycle where the digital image data corresponding to the displayed ultrasound image were obtained.

20. The method of claim 16, further comprising displaying cursors adjacent the ECG waveform indicative of the beginning and end of each heart cycle corresponding to the sections into which the digital image data was divided.

21. An ultrasound imaging system, comprising:
an ECG monitor operable to provide an ECG signal indicative of an ECG waveform;
an ECG processor coupled to receive the ECG signal from the ECG monitor, the ECG processor being operable to use the ECG signal to identify each of a plurality of heart cycles and to provide heart cycle data indicative of the beginning and end of each of the plurality of heart cycles;
an ultrasound probe having a plurality of transducer elements;
a transmitter coupled to the transducer elements of the ultrasound probe, the transmitter being operable to apply ultrasound signals to the transducer elements to cause the transducer elements to transmit ultrasound;
a receiver coupled to the transducer elements of the ultrasound probe, the receiver being operable to receive ultrasound echoes from the transmitted ultrasound, the receiver being operable to output echo signals corresponding to the ultrasound echoes;
a beamformer coupled to receive the echo signals from the receiver, the beamformer being operable to delay and sum the echo signals to form corresponding coherent echo signals;
a signal processor coupled to receive the coherent echo signals from the beamformer and the heart cycle data from the ECG processor, the signal processor being operable to process the coherent echo signals to form digital video data and to divide the digital video data into sections each of which corresponds to a respective ultrasound image obtained during a respective one of a plurality of heart cycles indicated by the heart cycle data; and
a digital video disk recorder coupled to receive the digital video data from the signals processor, the digital video disk recorder being operable to record a digital video disk containing the digital video data divided into chapters containing respective sections of the digital video data.

22. The ultrasound imaging system of claim 21 wherein the signal processor comprises:
a video processor coupled to receive the coherent echo signals from the beamformer, the video processor being operable to process the coherent echo signals to form image data; and
a streaming digital video processor coupled to receive the image data from the video processor, the streaming digital video processor being operable to convert the image data into streaming digital data divided into the sections indicated by the heart cycle data.

23. The ultrasound imaging system of claim 22 wherein the streaming digital video processor is coupled to receive signals indicative of the ECG waveform, the streaming digital video processor being operable to include in the streaming digital data ECG data corresponding to the ECU waveform divided into the sections indicated by the heart cycle data.

24. The ultrasound imaging system of claim 21 wherein the signal processor comprises a B-mode processor.

25. The ultrasound imaging system of claim 21 wherein the signal processor comprises a Doppler processor.
